(19) United States
(12) Patent Application Publication
(10) Pub. No.: US 2015/0120212 A1
(43) Pub. Date: Apr. 30, 2015

(54) METHOD FOR THE INVESTIGATION OF DIFFERENCES IN ANALYTICAL DATA AND AN APPARATUS ADAPTED TO PERFORM SUCH A METHOD

(71) Applicant: Micromass UK Limited, Wilmslow (GB)

(72) Inventors: Richard Denny, Staffordshire (GB); Kieran Neeson, Manchester (GB)

(73) Assignee: Micromass UK Limited, Wilmslow (GB)

(21) Appl. No.: 14/388,911

(22) PCT Filed: Mar. 28, 2013
(86) PCT No.: PCT/GB2013/050840

§ 371 Date: Sep. 29, 2014

(30) Foreign Application Priority Data
Mar. 30, 2012 (GB) 1205720.4

(51) Int. Cl. G01N 33/00; G01N 30/00

(52) U.S. Cl. CPC G01N 33/0004; G01N 30/00
USPC 702/24

(57) ABSTRACT

A method of investigating differences in data produced by at least one analytical instrument comprising providing a first data set from a first sample in a plurality of data bins; providing a second data set from a second sample in a plurality of data bins; providing a data model of said data sets in which the data in a plurality of data bins from the first data set is linked to the data in the corresponding bins of the second data set, each linked pair having an associated switch parameter linking the two together; and, exploring the posterior probability distribution for the data model as a function of the switch parameters to produce a posterior probability distribution map.

The present invention relates to method of investigating differences in data produced by at least one analytical instrument and apparatus adapted to perform the method. More particularly, but not exclusively, the present invention relates to the identification of differences in data sets using a data model for the data sets wherein the model ties the data in one bin in one data set to the data in the corresponding bin of the other data set.

The user of analytical instruments may wish to identify changes within two or more samples in various different applications. These applications may include, for example, reaction monitoring over time, quality control monitoring, patient diagnosis, and petrochemical investigative analysis.

It may be possible, particularly with relatively simple data, to identify changes by visibly looking at the data sets, and looking for differences. However, doing this can lead to incorrect identification of differences, due to various different factors. For example, these factors may include changes in conditions in which the analysis may be performed; and changes in concentration of the samples. These may lead to changes in the data produced by the instrument, which are not reflective of a change in the sample.

U.S. Pat. No. 8,012,764, and Proteinlynx global server (Micromass UK Limited) discloses a method where two separate samples are mass analysed and then the relative intensity, concentration or expression level of one or more components, molecules or analytes in a first sample is quantitated relative to the intensity, concentration or expression level of one or more components, molecules or analytes in a second sample.

This is performed by the identification of peaks in the data resulting from components within the first sample, and identification of the corresponding peaks from data relating to those same components in the second sample. The intensities of corresponding peaks are then interrogated to provide information relating to differences in the quantity of the components in both samples. However, the quantity of data produced by modern analytical instruments may be extremely large. This may result in the time taken for analysis of the data to be extremely long.

The method and apparatus according to the present invention seeks to overcome the problems of the prior art.

Accordingly in a first aspect the present invention provides a method of investigating differences in data produced by at least one analytical instrument comprising

providing a first data set from a first sample in a plurality of data bins;

providing a second data set from a second sample in a plurality of data bins;

providing a data model of said data sets in which the data in a plurality of data bins from the first data set is linked to the data in the corresponding bins of the second data set, each linked pair having an associated switch parameter linking the two together; and,

exploring the posterior probability distribution for the data model as a function of the switch parameters to produce a posterior probability distribution map.

The method according to the invention has the advantage of a considerable increase in speed and accuracy of processing the data. A further advantage of the method according to the invention is that not only are differences in the quantity of components within a sample identified, but components only present in one sample can also be identified.

Preferably at least one of the first and second data sets is a raw data set.

Preferably, each data bin represents a region of mass to charge ratio against mobility cell drift time.

The data held by each bin preferably relates to ion arrival count rate.

Preferably the switch parameter for each pair of linked data bins relates to the difference in the ion arrival count rate between the linked bins.

Preferably the data model models the ion arrival count rate for each bin as a product of a normalised count rate for that bin multiplied by a count rate scale factor for the data set to which the bin belongs, the switch parameter for each pair of linked bins relating to the difference in normalised ion count rate between the two bins.

Preferably, the step of exploring the posterior probability distribution further comprises exploring the posterior probability distribution as a function of the count rate scale factors of the data sets.

Preferably the switch parameter for each pair of linked data bins is a boolean parameter with one value corresponding to the same ion arrival count rate between the two linked bins and the other value corresponding to a different ion arrival count rate between the two bins.

Preferably, the data model further includes a parameter relating to the gain factor of the analytical instrument and the step of exploring the posterior probability distribution further comprises exploring the posterior probability distribution as a function of gain factor.

Preferably, the data model associates at least one shift correction with each bin, the step of exploring the posterior probability distribution further comprising exploring the posterior probability distribution as a function of the at least one shift correction.

Preferably the at least one shift correction is at least one of drift time, retention time, mass, precursor ion mass and product ion mass.

Optionally multiple data sets are provided from at least one of the first and second data samples.

Preferably, the method further comprises the step of identifying differences in data between the multiple data sets from the same sample to produce an estimate of variation in data produced by the at least one analytical instrument.

Optionally the posterior probability distribution is explored by a Monte Carlo algorithm.

The Monte Carlo algorithm may be a Markov Chain algorithm.

Preferably the Monte Carlo algorithm further comprises at least one sampling technique from the list comprising Gibbs sampling and Slice sampling.

Preferably the Monte Carlo algorithm further comprises at least one sampling technique from the list comprising Metropolis Hastings sampling and Nested sampling.

Optionally the method further comprises the step of analysing at least a portion of the explored region of the posterior probability distribution map to produce a result for at least one of the parameters of the data model.

Optionally the method further comprises the step of analysing at least a portion of the posterior probability distribution map to produce a map indicative of the differences between the data produced by the at least one analytical instrument from the first sample and the second sample.

Preferably, the method further comprises the step of further investigating the map indicative of the differences between the data produced by the at least one analytical instrument from the first sample and the second sample to determine differences in composition between the first and second samples.

Optionally the first data set and the second data set includes data produced by hydrogen deuterium exchange.

In a further aspect of the invention there is provided a computer program element comprising computer readable code means for causing a processor to implement the method of any of claims 1-21.

Preferably the computer program element is embodied on a computer readable medium.

In a further aspect of the invention there is provided a computer readable medium having a program stored thereon, wherein the program is adapted to make a computer execute a procedure to implement the method of any of claims 1-21.

In a further aspect of the invention there is provided an analytical instrument, adapted to perform the method as claimed in any one of claims 1-21.

The present invention will now be described by way of example only, and not in any limitative sense with reference to the accompanying drawings in which

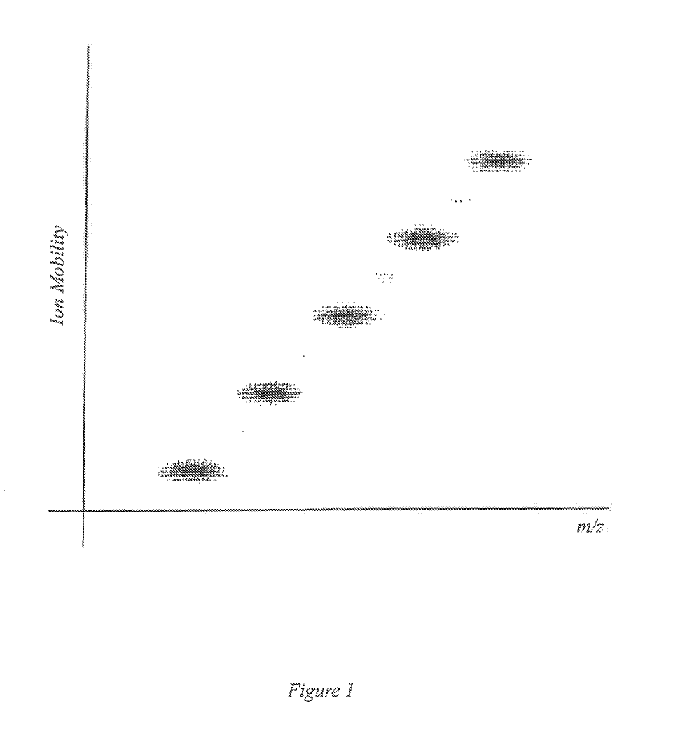

FIG. 1 shows raw data produced by an analytical instrument.

Shown in FIG. 1 is raw data produced by an analytical instrument, in this case a mass spectrometer. The data may be viewed as a data set comprising a plurality of bins which in this embodiment are arranged in a rectangular array. The x axis of the array is mass to charge ratio (m/z). The y axis is mobility cell drift time. Each bin therefore represents a small area of mass to charge ratio and mobility cell drift time. Contained within each bin is a count of the number of ion arrivals within a predetermined time (the ion arrival count rate). In practice a detector response is recorded for each bin which may be taken to be proportional to the count. The production of such data sets from analytical instruments is known and will not be discussed in further detail.

In non-limiting outline, the purpose of the method according to the invention is to is to compare two similar data sets to obtain a bin by bin probability map that gives the probability that the counts ascribed to corresponding bins arise from the same Poisson source.

In a first step of an embodiment of a method according to the invention a first data set from a first sample is provided. The first data set (image) is arranged in a plurality of data bins as described above.

In a second step a second data set from a second sample is provided. The second data set (image) is also arranged in a plurality of bins as described above.

In order to compare the two data sets and identify differences between them a data model is provided. The data model comprises two data arrays of bins corresponding to the bins of the two data sets.

The i'th bin (where i indexes the corresponding bins in the two images) is assigned a Poisson ion arrival rate λ₁ᵢ in the first image, which is split into two factors, λ₁ᵢ=μ₁ᵢσ₁. Here, μ₁ᵢ is exponentially distributed with unit mean,

Pr(μ₁ᵢ)=e^{-μ}^{₁ᵢ},

with an identically distributed rate μ₂ᵢ in the second image. Scaling by the count rate scale factor σ₁ (or σ₂ in the second image) allows patterns of rates μ₁ and μ₂ to be established independent of the scale factors required to achieve agreement with the data. The ion count η₁ᵢ in the bin image 1 has probability

$$Pr ( {n}_{1 i} \vert {μ}_{1 i} , {σ}_{1} ) = \frac{{{e}^{- {σ}_{1} {μ}_{1 i}} ( {σ}_{1} {μ}_{1 i} )}^{{n}_{1 i}}}{{n}_{1 i} !}$$

and the joint probability is

$$Pr ( {n}_{1 i} , {μ}_{1 i} \vert {σ}_{1} ) = \frac{{{e}^{- ( {σ}_{1} + 1 ) {μ}_{1 i}} ( {σ}_{1} {μ}_{1 i} )}^{{n}_{1 i}}}{{n}_{1 i} !} .$$

one can marginalise over (integrate out) μ₁ᵢ to give

$$Pr ( {n}_{1 i} \vert {σ}_{1} ) = \frac{{σ}_{1}^{{n}_{1 i}}}{{( {σ}_{1} + 1 )}^{{n}_{1 i} + 1}} .$$

If the corresponding bins in the two images are assumed to have the same Poisson rate so that μ₁ᵢ=μ₂ᵢ then μᵢ may again be marginalised away to give

$$Pr ( {n}_{1 i} , {n}_{2 i} \vert {σ}_{1} , {σ}_{2} , {μ}_{1 i} = {μ}_{2 i} ) = \frac{{σ}_{1}^{{n}_{1 i}} {σ}_{2}^{{n}_{2 i}}}{{( {σ}_{1} + {σ}_{2} + 1 )}^{{n}_{1 i} + {n}_{2 i} + 1}} \frac{( {n}_{1 i} + {n}_{2 i} ) !}{{n}_{1 i} ! {n}_{2 i} !} .$$

Conversely, if they are assumed to have different rates μ₁ᵢ and μ₂ᵢ are marginalised separately to give

$$Pr ( {n}_{1 i} , {n}_{1 i} \vert {σ}_{1} , {σ}_{2} , {μ}_{1 i} \neq {μ}_{2 i} ) = \frac{{σ}_{1}^{{n}_{1 i}}}{{( {σ}_{1} + 1 )}^{{n}_{1 i} + 1}} \frac{{σ}_{2}^{{n}_{2 i}}}{{( {σ}_{2} + 1 )}^{{n}_{2 i} + 1}} .$$

From these equations the posterior probability distribution for the data model can be derived.

The basic scheme is to explore the parameters of the data model by constructing an ergodic Markov chain whose stationary distribution is the joint probability distribution of data and model parameters. The chain is constructed using transitions for each parameter which leaves this desired distribution invariant. The chain will be ergodic if, for each transition, the probability distribution is greater than zero. This ensures that there is a non-zero probability of accessing any state, after iterating over each parameter starting from any initial state.

One wishes to obtain a posterior probability distribution map of Pr({μ₁ᵢ≠μ₂ᵢ}¦image 1={η₁ᵢ}, image 2={η₂ᵢ}) and so must explore the space of same/different assignment of rates for each bin along with the two scale factors σ₁ and σ₂ for each image.

Let βᵢ be a switch state linking a bin in one data set to a corresponding bin in the other data set. Let βᵢ≡(μ₁ᵢ=μ₂ᵢ) be a Boolean variable. A transition from βᵢ=false to βᵢ=true has a probability ratio

$$\frac{Pr ( {β}_{i} = true \vert {σ}_{1} , {σ}_{2} )}{Pr ( {β}_{i} = false \vert {σ}_{1} , {σ}_{2} )} = \frac{Pr ( {β}_{i} = true )}{Pr ( {β}_{i} = false )} \frac{{( {σ}_{1} + 1 )}^{{n}_{1 i} + 1} {( {σ}_{2} + 1 )}^{{n}_{2 i} + 1}}{{( {σ}_{1} + {σ}_{2} + 1 )}^{{n}_{1 i} + {n}_{2 i} + 1}} \frac{( {n}_{1 i} + {n}_{1 i} ) !}{{n}_{1 i} ! {n}_{2 i} !} .$$

These transitions can be explored by a Gibbs sampling scheme whereby a transition to βᵢ=b is made where

$$b = \frac{Pr ( {β}_{i} = true \vert {σ}_{1} , {σ}_{2} )}{Pr ( {β}_{i} = false \vert {σ}_{1} , {σ}_{2} )} > \frac{r}{1 - r} ,$$

and r is a random number drawn from (0,1).

Once such a posterior probability map has been determined it can be analysed to produce a variety of results. By sampling appropriate states in the map one can derive an average value of the switch state for one or more bins. For bins where the average value is close to false this indicates likelihood of a significant difference in data from the two samples in that bin, so indicating a difference in composition of the two samples. For bins where the average value of the switch state is close to false this suggests no difference in composition. One can perform this analysis for each bin to produce a map which is indicative of the differences between the data produced by the at least one analytical instrument from the first sample and the second sample. This in turn can be used to determine differences in composition between the first sample and the second sample. Typically when sampling states in the posterior probability map one tends not to take into consideration the first few determined states in the chain. This is because the initial starting state of the model may be a highly improbable state and it takes a few iterations to approach the region of probable states.

In the above embodiment the posterior probability distribution is explored as a function of the switch state β. In more complex models the posterior probability distribution can be explored as a function of further variables.

One particular further variable is the count rate scale factor. The likelihood for scale factors σ₁ or σ₂ is

$$Pr ( \{ {n}_{1 i} \} , \{ {n}_{2 i} \} \vert {σ}_{1} , {σ}_{2} , \{ {β}_{i} \} ) ∝ \frac{{σ}_{1}^{{\sum }_{i} {n}_{1 i}} {σ}_{2}^{{\sum }_{i} {n}_{2 i}}}{{( {σ}_{1} + {σ}_{2} + 1 )}^{{\sum }_{{β}_{j} = true} {n}_{1 j} + {n}_{2 j} + 1} {( {σ}_{1} + 1 )}^{{\sum }_{{β}_{k} = false} {n}_{1 k} + 1} {( {σ}_{2} + 1 )}^{{\sum }_{{β}_{k} = false} {n}_{2 k} + 1}} .$$

Probable values for σ₁ and σ₂ may easily be explored by slice sampling, once a suitable prior has been established. It is straightforward to decouple slice sampling from the details of a prior probability distribution for θ for instance by driving it through a controlling variable u uniformly distributed on (0,1) such that

$$u ( Θ ) = {\int }_{0}^{Θ} Pr ( θ ) d θ .$$

A convenient and unrestrictive prior for σ is

$$Pr ( σ \vert S ) = \frac{S}{{( S + σ )}^{2}} ,$$

so that

$$u ( σ ) = \frac{σ}{S + σ}$$

$$and$$

$$σ ( u ) = \frac{Su}{1 - u}$$

where S is the median value.

Once one has determined the posterior probability distribution map as a function of σ one can analyse it by sampling appropriate states to determine the likely values of σ for the two data sets.

A further suitable variable is the gain factor. The response of the detector x of the analytical instrument may be proportional to the ion arrival count n rather than identical to it and one may be uncertain about the constant of proportionality or gain factor γ. The factor can be included in the above analysis by scaling down σ and S by γ so that the prior on a becomes

$$Pr ( σ \vert S , γ ) = \frac{S / γ}{{( S / γ + σ )}^{2}}$$

and by modifying the Poisson likelihood to the form

$$Pr ( x \vert μ , σ , γ ) = \frac{{{e}^{- σ u} ( σμ )}^{n}}{γ n !} ,$$

where [x/Y]=n. The complete likelihood over all pixels (bins) is

$$Pr ( \{ {x}_{1 i} \} , \{ {x}_{2 i} \} \vert \{ {β}_{i} \} , {σ}_{1} , {σ}_{2} , γ ) = {γ}^{- 2 I} {σ}_{1}^{{\sum }_{i} {n}_{1 i}} {σ}_{2}^{{\sum }_{i} {n}_{2 i}} \times {\prod }_{{β}_{j} = true} \frac{( {n}_{1 j} + {n}_{2 j} ) !}{{n}_{1 j} ! {n}_{2 j} ! {( {σ}_{1} + {σ}_{2} + 1 )}^{{n}_{1 j} + {n}_{2 j} + 1}} \times {\prod }_{{β}_{k} = false} \frac{1}{{( {σ}_{1} + 1 )}^{{n}_{1 k} + 1} {( {σ}_{2} + 1 )}^{{n}_{2 k} + 1}} ,$$

where I is the total number of pixels (bins) in an image. A similar prior for γ may be employed to that used for the scale factors σ but offset by one as the lowest attainable gain corresponds to the counting of single ions. Slice sampling may again be employed to generate transitions.

Often there is some shift in the drift time calibration between acquisitions, the shift being of the order of one bin (of typically 200). Each image may be shifted and re-sampled onto a common drift time axis, from where the likelihood can be re-computed and explored with slice sampling. There are a couple of technicalities involved in this procedure. Firstly, the first and last points on the drift time axis are not shifted so that the total number of counts is conserved when data are resampled. For safety the extremities are placed away from the interior values by a large margin. Secondly, once all images have been shifted the common axis may be shifted to relax the image shifts against their combined prior probability. A Gaussian prior with a standard deviation of one or two bins typically reflects the drift time variability adequately. Slice sampling may again be employed to generate transitions.

There is some variation between acquisitions of nominally equivalent samples beyond strict application of Poisson statistics. These replicate acquisitions may be used to accommodate extra variation between nominally non-equivalent samples that may not be significant. Consider C replicate injections of sample 1 (the control) and A replicate injections of sample 2 (the analyte) giving a total of C+A=R images. The complete likelihood becomes

$$Pr ( \{ {x}_{ri} \} \vert \{ {β}_{i} \} , \{ {σ}_{r} \} , γ ) = {γ}^{- RI} {\prod }_{r} {{σ}_{r}^{{\sum }_{i} {n}_{ri}} ( {\prod }_{i} {n}_{ri} ! )}^{- 1} \times {\prod }_{{β}_{j} = true} \frac{( {\sum }_{r} {n}_{rj} ) !}{{( 1 + {\sum }_{r} {σ}_{r} )}^{1 + {\sum }_{r} {n}_{rj}}} \times {\prod }_{{β}_{k} = false} \frac{( {\sum }_{c} {n}_{ck} ) !}{{( 1 + {\sum }_{c} {σ}_{c} )}^{1 + {\sum }_{c} {n}_{ck}}} \frac{( {\sum }_{a} {n}_{ak} ) !}{{( 1 + {\sum }_{a} {σ}_{a} )}^{1 + {\sum }_{a} {n}_{ak}}} ,$$

Where subscripts c, a and r indicate members of the control group, analyte group and entire group respectively.

The probability ratios for the switch states β and scale factors σ are easily modified as in the above to accommodate the control and analyte groupings.

The above analysis depends on the image pixel (bin) size. At both extremes (single pixel per image and single count or zero counts per pixel) the data becomes uninformative with regard to the model being applied. Choice of pixel size is therefore an important consideration.

The method according to the invention has been described in various embodiments above with reference to Slice sampling and Gibbs sampling. Other sampling methods may be employed more particularly but not limited to Metropolis Hastings sampling and Nested sampling.

The first data set and second data set can include data produced by hydrogen deuterium exchange.

The method and apparatus of the invention may be used to monitor samples in a batch control process, wherein samples may be compared to a predetermined standard, to ascertain whether, and by how much, and in what components, the sample deviates from the standard. This is of use in, for example, the assessment of petroleum and biofuel samples, which must adhere to strict standards.

The method and apparatus of the invention may further be used in a sequential process. Rather than comparing a sample against a fixed standard, the samples may be compared against an earlier sample. This is of use in, for example, monitoring drug metabolism over time, or the degradation of petroleum over time. Also for the relative comparison of materials in the chemical industry such as polymers and formulated blends such as paints, coatings, sealants, cosmetics and agrochemicals. Here the method and apparatus of invention are used to detect and characterise composition changes resulting from varying reaction conditions, errors in formulation make-up, degradation and ageing of materials as a result of environmental conditions and/or mechanical use.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A method of investigating differences in data produced by at least one analytical instrument comprising
providing a first data set from a first sample in a plurality of data bins;
providing a second data set from a second sample in a plurality of data bins;
providing a data model of said data sets in which the data in a plurality of data bins from the first data set is linked to the data in the corresponding bins of the second data set, each linked pair having an associated switch parameter linking the two together; and,
exploring the posterior probability distribution for the data model as a function of the switch parameters to produce a posterior probability distribution map.

2. A method as claimed in claim 1, wherein at least one of the first and second data sets is a raw data set.

3. A method as claimed in claim 1, wherein each bin represents a region of mass to charge ratio against mobility cell drift time.

4. A method as clamed in claim 1, wherein the data held by each bin relates to ion arrival count rate.

5. A method as claimed in claim 4, wherein the switch parameter for each pair of linked data bins relates to the difference in the ion arrival count rate between the linked bins.

6. A method as claimed in claim 5, wherein the data model models the ion arrival count rate for each bin as a product of normalised count rate for that bin multiplied by a count rate scale factor for the data set to which the bin belongs, the switch parameter for each pair of linked bins relating to the difference in normalised ion count rate count rate between the linked bins.

7. A method as claimed in claim 6, wherein the step of exploring the posterior probability distribution further comprises exploring the posterior probability distribution as a function of the count rate scale factors of the data sets.

8. A method as claimed in claim 5, wherein the switch parameter for each pair of linked data bins is a boolean parameter with one value corresponding to the same ion arrival count rate between the two linked bins and the other value corresponding to a different ion arrival count rate between the two bins.

9. A method as claimed in claim 1, wherein the data model further includes a parameter relating to the gain factor of the analytical instrument and the step of exploring the posterior probability distribution further comprises exploring the posterior probability distribution as a function of gain factor.

10. A method as claimed in claim 1, wherein the data model associates at least one shift correction with each bin, the step of exploring the posterior probability distribution further comprising exploring the posterior probability distribution as a function of the at least one shift correction.

11. A method as claimed in claim 10, wherein the at least one shift correction is at least one of drift time, retention time, mass, precursor ion mass and product ion mass.

12. A method as claimed in claim 1, wherein multiple data sets are provided from at least one of the first and second data samples.

13. A method as claimed in claim 12 further comprising the step of identifying differences in data between the multiple data sets from the same sample to produce an estimate of the variation in data produced by the at least one analytical instrument.

14. A method as claimed in claim 1, wherein the posterior probability distribution is explored by a Monte Carlo algorithm.

15. A method as clamed in claim 14, wherein the Monte Carlo algorithm is a Markov Chain algorithm.

16. A method as claimed in claim 14, wherein the Monte Carlo algorithm further comprises at least one sampling technique from the list comprising Gibbs sampling, Slice sampling, Hastings sampling, and Nested sampling.

17. (canceled)

18. A method as claimed in claim 1, further comprising the step of analysing at least a portion of the posterior probability distribution map to produce a result for at least one of the parameters of the data model.

19. A method as claimed in claim 1, further comprising the step of analysing at least a portion of the posterior probability distribution map to produce a map indicative of the differences between the data produced by the at least one analytical instrument from the first sample and the second sample.

20. A method as claimed in claim 19, further comprising further investigating the map indicative of differences between the data produced by the at least one analytical instrument from the first sample and the second sample to determine differences in composition between the first and the second samples.

21. A method as claimed in any claim 1, wherein the first data set and the second data set includes data produced by hydrogen deuterium exchange.

22-25. (canceled)
